(19) United States
(12) Patent Application Publication
(10) Pub. No.: US 2026/0108431 A1
(43) Pub. Date: Apr. 23, 2026

(54) BIOACTIVE COMPOSITES FOR BONE VOID FILLER COMPOSITIONS AND METHODS

(71) Applicant: Orthomod LLC, Dayton, OH (US)

(72) Inventors: David Louis Kirschman, Dayton, OH (US); Dewey J. Weeda, Oakwood, OH (US); Shawn A. Hunter, Springboro, OH (US)

(21) Appl. No.: 19/339,506

(22) Filed: Sep. 25, 2025

Related U.S. Application Data
(60) Provisional application No. 63/710,756, filed on Oct. 23, 2024.
(60) Provisional application No. 63/710,763, filed on Oct. 23, 2024.

(51) Int. Cl. A61K 6/802; A61K 6/15; A61K 6/838

(52) U.S. Cl. CPC A61K 6/802; A61K 6/15; A61K 6/838

(57) ABSTRACT

This invention relates to bioactive composites, and more particularly, to bone void filler compositions including bioactive composites and/or implants and methods for the promotion of bone growth and regeneration. This invention also relates to bone void fillers, specifically to a high-strength calcium acrylic modified polymer (CAMP) material designed for orthopedic and surgical applications requiring both structural support and enhanced biological properties.

### CROSS REFERENCE TO RELATED APPLICATIONS

This application claims the benefit of U.S. Provisional Application Ser. No. 63/710,756, filed on Oct. 23, 2024, and U.S. Provisional Application Ser. No. 63/710,763, filed on Oct. 23, 2024; the disclosures of which are incorporated herein by reference and made a part hereof.

### BACKGROUND OF THE INVENTION

### 1. Field of the Invention

This invention relates to bioactive composites, and more particularly, to bone void filler compositions including bioactive composites and/or implants and methods for the promotion of bone growth and regeneration. This invention also relates to bone void fillers, specifically to a high-strength calcium acrylic modified polymer (CAMP) material designed for orthopedic and surgical applications requiring both structural support and enhanced biological properties.

### 2. Description of the Related Art

Bone void fillers are materials which are used to fill gaps or voids in genuine or artificial bones or implants from trauma, surgery, or disease. Bone void fillers provide a scaffold or support on the damaged portion of the bone to help the bone's natural healing process. Bone void fillers can be made of a variety of products including synthetic, natural, or composite materials.

Bone void fillers currently available in the medical field often fail to provide adequate mechanical strength while promoting biological healing. There is a critical need for materials that can withstand physiological loading conditions while facilitating bone regeneration, particularly in load-bearing applications or large segmental defects. Current solutions typically require additional fixation devices and often lack the biological properties necessary for optimal bone regeneration.

The synthetic bone void fillers can include substances like hydroxyapatite, calcium sulfate, and bioactive glass. The natural bone void fillers can be allografts, which are derived from humans, or xerographs, which are derived from animals. The composite material can be a combination of the synthetic and natural bone void filler 16 which allow for enhanced properties and effectiveness.

Nevertheless, the bone void fillers currently available in the medical field often fail to provide adequate mechanical strength to the damaged bone while promoting biological healing. The current bone void fillers do not have the attributes to withstand the physiological loading conditions while facilitating bone regeneration, which is particularly apparent when the damaged bone is in a load-bearing bone or the damaged bone has large segmental defects. When the current bone void fillers lack the mechanical strength to support the damaged bones, additional fixation devices are required. These fixation devices are commonly made of material such as metal, which lack the biological properties necessary for optimal bone regeneration.

U.S. Patent Publication No. 20210022872 relates to a system, device/implant, method and processes for manufacturing a filament and an implant having at least one or a plurality of areas in the implant comprised of selectively-place bone to facilitate osteoconductivity and, potentially, osteoinductivity after the implant is implanted into a patient.

U.S. Patent Publication No. 20220296780 relates to a method of generating a bioceramic-containing biomaterial-derived thermoplastic extrusion is provided. The method includes combining a bioceramic-containing solid with at least one thermoplastic resin, wherein the bioceramic-containing solid is uniformly dispersed in the resin. The method further includes extruding the bioceramic-containing solid included in the resin to create a net shape. The net shape is selected from a group consisting of a filament, a pellet, a bar, a molding, and a three-dimensional printing material stock.

U.S. Patent Publication No. 20250025603 relates to surgical implants and methods of manufacturing the implants. The surgical implant includes an outer body made of a polymer, at least one internal strut coupled to the outer body, where the internal strut has a smaller diameter than the outer body, and ceramic granules deposited on the surface of the internal strut. Methods of manufacturing a surgical implant generally include injecting a molten mixture containing a polymer and ceramic particles into a mold. The mold defines a negative space for forming the implant. As the molten mixture flows through the negative space, the mold constricts the flow at the internal strut, causing the ceramic granules to deposit on the surface of the internal strut. The mixture is then cooled to form the implant, with the ceramic granules at least partially exposed on its surface.

U.S. Patent Publication No. 20250041027 relates to surgical implants and methods of manufacturing the implants. The implants are generally osteoconductive composite implants utilizing minimally-crosslinked acrylic carriers combined with osteoconductive ceramics. The methods generally involve dissolving a monomer and an initiator in a solvent to form a reaction mixture, controlling the temperature and viscosity of the reaction mixture to allow solution polymerization, polymerizing the monomer in the reaction mixture to form an acrylic carrier, recovering the acrylic carrier, extruding the acrylic carrier with an osteoconductive ceramic to form an osteoconductive biomaterial, and processing the osteoconductive biomaterial to form the osteoconductive composite implant.

### SUMMARY OF THE INVENTION

The present invention provides a novel CAMP-based bone void filler that combines superior mechanical properties with enhanced biological activity. The material can be delivered in various forms including granules, shapes, injectable formulations, and putty preparations, making it versatile for different clinical applications. The invention addresses the longstanding need for a bone void filler that can withstand physiological loading while actively promoting bone regeneration.

In one aspect, one embodiment of the invention comprises a bioactive composite comprising an osteoconductive ceramic compound dispersed within an acrylic polymer matrix; wherein the acrylic polymer matrix comprises one or more polymerized acrylic compounds.

In another aspect, another embodiment of the invention comprises a method of structurally supporting a bone defect including defects formed from trauma and surgery, the method comprising providing an injectable bone void filler composition comprising an osteoconductive ceramic compound dispersed within an acrylic polymer matrix, wherein the acrylic polymer matrix comprises one or more polymerized acrylic compounds; and a carrier; injecting the injectable bone void filler composition to the bone defect and allowing the injectable bone void filler composition to harden in situ to thereby form a structural support to the bone.

In another aspect, another embodiment of the invention comprises a method of manufacturing a dental implant, the method comprising molding a body of the dental implant using a bioactive composite comprising an osteoconductive ceramic compound dispersed within an acrylic polymer matrix, wherein the acrylic polymer matrix comprises one or more polymerized acrylic compounds, and wherein the acrylic polymer matrix is crosslinked through one or more of radiation-induced crosslinking, temperature-induced crosslinking, solution polymerization, di-allyl linking, and chemical crosslinking; treating a surface of the body to thereby form a plurality of surface pores in the surface.

In still another aspect, another embodiment of the invention comprises a dental implant comprising a body formed from a calcium acrylic modified polymer (CAMP), comprising a polymerized acrylic compounded with calcium-containing ceramics wherein the implant is configured for insertion into bone tissue; the surface of the body comprising microporosity to enhance osseointegration by promoting bone tissue ingrowth into the porous structure of the implant; said acrylic comprising one or more of radiation-induced crosslinking, temperature-induced crosslinking, solution polymerization, di-allyl linking, or chemical crosslinking.

In another aspect, one embodiment of the invention comprises a method of manufacturing a dental implant, comprising molding a body of the implant from ceramic-acrylic modified polymer (CAMP); introducing surface microporosity of the body of the implant to promote osseointegration, wherein the CAMP material is designed to enhance bone tissue interaction and ensure faster healing and stronger integration compared to traditional implant materials.

In yet another aspect, one embodiment of the invention comprises an injectable bone void filler comprising a ceramic acrylic modified polymer (CAMP) material; wherein the CAMP material is configured to provide structural support to the bone void and serves as a scaffold for bone regrowth; wherein the material maintains mechanical integrity until natural bone regenerates within the void; said acrylic material comprising poly methyl methacrylate, Ethylene glycol dimethacrylate (EGDMA), Triethylene glycol dimethacrylate (TEGDMA), Poly(ethylene glycol) dimethacrylate (PEGDMA), N,N′-methylenebisacrylamide (MBA), Allyl methacrylate (AMA), Urethane dimethacrylate (UDMA), Bisphenol A glycerolate dimethacrylate (Bis-GMA), Glyceryl dimethacrylate (GDMA).

In another aspect, one embodiment of the invention comprises a method for treating bone voids, comprising injecting a flowable CAMP-based material into a bone void and allowing the material to harden in situ, wherein the material provides structural support and serves as a scaffold for new bone growth.

In another aspect, one embodiment of the invention comprises a method of treating large bone defects, comprising selecting an appropriate form of the CAMP material based on defect characteristics, combining the material with a carrier if required, and implanting the material in conjunction with standard fixation hardware, wherein the material provides both immediate mechanical stability and long-term biological integration.

This invention, including all embodiments shown and described herein, could be used alone or together and/or in combination with one or more of the features covered by one or more of the following list of features:
The bioactive composite wherein the one or more polymerized acrylic compounds are selected from the group consisting of poly methyl methacrylate, ethylene glycol dimethacrylate (EGDMA), triethylene glycol dimethacrylate (TEGDMA), poly(ethylene glycol) dimethacrylate (PEGDMA), N,N′-methylenebisacrylamide (MBA), allyl methacrylate (AMA), urethane dimethacrylate (UDMA), bisphenol A glycerolate dimethacrylate (Bis-GMA), glyceryl dimethacrylate (GDMA), and mixtures thereof.
The bioactive composite wherein the osteoconductive ceramic compound is selected from the group consisting of hydroxyapatite (HA, Ca₁₀(PO₄)₆(OH)₂), beta-tricalcium phosphate (β-TCP, Ca₃(PO₄)₂), biphasic calcium phosphate (BCP, combination of HA and β-TCP), calcium sulfate (CaSO₄), tetracalcium phosphate, octacalcium phosphate, bioactive glasses, silicate-based bioactive glasses, borate-based bioactive glasses, and combinations thereof.
The bioactive composite wherein the acrylic polymer matrix is cross-linked.
The bioactive composite wherein the cross-linked acrylic polymer matrix comprises a plurality of pores wherein an average diameter of each pore of the plurality of pores is from 1 μm to 500 μm.
The bioactive composite wherein the average diameter of each pore of the plurality of pores is from 1 μm to 50 μm.
The bioactive composite wherein the cross-linked acrylic polymer matrix structure is stable for a period of at least 3 months.
The bioactive composite wherein the bioactive composite has a compressive strength of 10 MPa or greater.
The bioactive composite wherein the bioactive composite is radiopaque.
The bioactive composite further comprising one or more bioactive agents selected from the group consisting of growth factors, stem cell attractants, osteogenic proteins, and combinations thereof.
The bioactive composite further comprising one or more of antimicrobial agents, analgesics, and growth factors, with and without controlled release properties.
The bioactive composite wherein an injectable bone void filler composition is in a flowable form; and a carrier.
The injectable bone void filler composition wherein the carrier is selected from the group consisting of one of hyaluronic acid, collagen solutions, chitosan, sodium alginate, gelatin, methylcellulose, carboxymethylcellulose (CMC), polyethylene glycol (PEG) solutions, Pluronic F-127 (poloxamer 407), poly(vinyl alcohol) (PVA), poly(N-isopropylacrylamide) (PNIPAM), PEG-PCL copolymers, poly(lactide-co-glycolide) (PLGA) solutions, polycaprolactone (PCL) solutions, poly(L-lactide) (PLLA) solutions, poly(D,L-lactide) (PDLLA) solutions, poloxamer-based hydrogels, chitosan derivatives, glycerol, dextran, beta-cyclodextrin solutions, hydroxypropyl methylcellulose (HPMC), phosphate buffered saline (PBS) with thickening agents, and combinations thereof.
The injectable bone void filler composition wherein the bioactive composite further comprises one or more bioresorbable components.
The injectable bone void filler composition wherein the injectable bone void filler is thixotropic.
The injectable bone void filler composition wherein the injectable bone void filler composition has a set time of from about 5 minutes to about 30 minutes.
The bioactive composite wherein a dental implant comprises a body comprising the bioactive composite.
The dental implant wherein the body comprises a surface, wherein the surface includes a plurality of pores.
The dental implant wherein the body further comprises a bioactive coating over the surface, and wherein the bioactive coating is selected from the group consisting of osteoconductive ceramics, collagen, growth factors, and combinations thereof.
The dental implant wherein the surface microporosity has an average pore size between 1 μm and 100 μm to optimize bone tissue interaction and promote rapid osseointegration.
The dental implant wherein the CAMP material comprises one or more osteoconductive ceramics, including on or more of calcium phosphate, tricalcium phosphate, hydroxyapatite, or bioglass, integrated into an acrylic polymer matrix to provide bioactivity and mechanical properties that mimic natural bone.
The dental implant wherein the CAMP material is formulated to promote bone healing by enhancing cellular adhesion, proliferation, and differentiation of osteoblasts on the implant surface, including osteoblast-mediated surface remodeling and bone formation to a depth of greater than 1 mm from the implant surface.
The dental implant wherein the CAMP material is biocompatible and designed to reduce the risk of peri-implantitis by providing long-term stability and resistance to bacterial colonization.
The dental implant wherein the body of the implant is shaped and dimensioned to match the anatomical requirements of a dental implant site, including a screw-type design for insertion into the mandible.
The dental implant wherein the surface microporosity is created by gas entrapment during a molten acrylic phase, soluble porogen, surface chemical etching, or other surface modification techniques to control the size and distribution of pores.
The dental implant further comprising a bioactive coating or external surface of a multi-part assembly applied to the microporous surface, wherein the coating is selected from the group consisting of osteoconductive ceramics, collagen, and growth factors, to further enhance osseointegration and bone healing.
The dental implant wherein the CAMP material is modified to include analgesics or antimicrobial agents, such as silver ions or antibiotics.
The dental implant wherein the porosity of the surface extends into the internal structure of the implant, forming interconnected pores that allow for vascularization and enhanced bone ingrowth.
The dental implant wherein the CAMP material is engineered to provide a modulus of elasticity that closely matches that of natural bone, thereby minimizing stress shielding effects and improving implant stability.
The dental implant wherein the CAMP material is designed to maintain long-term structural stability and resist degradation or wear under normal physiological loads experienced in the oral cavity.
The method wherein the surface microporosity is created by molten-state gas entrapment, porogen, or chemical etching techniques, thereby producing a porous structure optimized for bone ingrowth.
The method wherein the CAMP material is synthesized by incorporating osteoconductive ceramic into an acrylic polymer matrix, resulting in a composite material that mimics the mechanical and biological properties of bone.
The dental implant wherein the CAMP material has a radiodensity similar to bone, allowing for improved radiographic visibility during post-operative monitoring and ensuring proper implant placement and integration.
The dental implant wherein the CAMP material is designed to promote sustained ion release, including calcium and phosphate ions, to stimulate bone mineralization around the implant.
The dental implant wherein the CAMP material and/or component is compatible with other dental prosthetics and abutments, including mechanical assembly with metallic or polymer components, enabling integration into a variety of restorative dental procedures.
The bone void filler wherein the CAMP material comprises a combination of at least one of Hydroxyapatite (HA, Ca₁₀(PO₄)₆(OH)₂), Beta-tricalcium phosphate (β-TCP, Ca₃(PO₄)₂), Biphasic calcium phosphate (BCP, combination of HA and β-TCP), Calcium sulfate (CaSO₄), Tetracalcium phosphate, Octacalcium phosphate, Bioactive glasses, Silicate-based bioactive glasses, Borate-based bioactive glasses, Glass-ceramics: calcium phosphate and an acrylic polymer matrix, providing both bioactivity and strength suitable for load-bearing applications.
The bone void filler wherein the material is designed to be injectable or flowable within a carrier such as Hyaluronic acid, Collagen solutions, Chitosan, Sodium alginate, Gelatin, Methylcellulose, Carboxymethylcellulose (CMC), Polyethylene glycol (PEG) solutions, Pluronic F-127 (poloxamer 407), Poly(vinyl alcohol) (PVA), Poly(N-isopropylacrylamide) (PNIPAM), PEG-PCL copolymers, Poly(lactide-co-glycolide) (PLGA) solutions, Polycaprolactone (PCL) solutions, Poly(L-lactide) (PLLA) solutions, Poly(D,L-lactide) (PDLLA) solutions, Poloxamer-based hydrogels, chitosan derivatives, Glycerol, Dextran, Beta-cyclodextrin solutions, Hydroxypropyl methylcellulose (HPMC), and/or Phosphate buffered saline (PBS) with thickening agents.
The bone void filler wherein the material is bioactive and promotes bone cell adhesion, proliferation, and differentiation by releasing calcium and/or phosphate ions into the surrounding tissue.
The bone void filler wherein the material forms a porous structure upon hardening, allowing for vascularization and infiltration of new bone cells into the scaffold.
The bone void filler wherein the material is designed to gradually degrade over time as new bone tissue forms, ensuring the complete replacement of the filler by natural bone.
The bone void filler wherein the CAMP material is radiopaque, allowing for easy monitoring of the filler placement and bone regeneration using radiographic imaging.
The bone void filler wherein the CAMP material exhibits a compressive strength of at least 10 MPa, sufficient to provide mechanical support in load-bearing bone voids.
The bone void filler wherein the material is designed to maintain its structural integrity for a period of 3 to 12 months, providing temporary support until the bone fully regenerates.
The bone void filler wherein the material is modified with bioactive agents selected from the group consisting of growth factors, stem cell attractants, and osteogenic proteins to enhance bone healing.
The bone void filler wherein the CAMP material includes micropores with an average size between 1 μm and 50 μm to facilitate bone cell ingrowth and nutrient exchange.
The bone void filler wherein the material is suitable for use in orthopedic trauma, tumor resections, or other conditions requiring bone reconstruction.
The method wherein the CAMP material is injected through a minimally invasive surgical procedure to reduce patient recovery time and minimize tissue damage.
The bone void filler wherein the material is formulated to exhibit thixotropic properties, allowing for easy injection and flow into irregularly shaped bone voids.
The bone void filler wherein the material includes antimicrobial agents to prevent infection at the surgical site or analgesics.
The bone void filler wherein the CAMP material is combined with bioresorbable fibers or particles to further enhance mechanical properties and ensure a controlled degradation profile.
The bone void filler wherein the material is biocompatible and resists inflammation or immune rejection when implanted in a human body.
The bone void filler wherein the CAMP material is designed to set within a time frame of 5 to 30 minutes after injection, providing rapid mechanical stabilization to the bone void.
The bone void filler wherein the material is capable of forming a cohesive bond with the surrounding bone tissue, preventing migration or displacement of the filler after implantation.
The bone void filler wherein the material is designed for use in pediatric patients, providing structural support while allowing for natural bone growth and development over time.
The bone void filler wherein the bone reconstruction system comprises a CAMP-based injectable bone void filler and an applicator configured to inject the flowable material into a bone void, wherein the system provides structural support and bioactivity for the treatment of bone defects caused by trauma or surgery.
The bone void filler wherein the material demonstrates fatigue resistance capable of withstanding cyclic loading of at least 1 million cycles under physiological loading conditions.
The bone void filler wherein the material is provided in the form of granules ranging from 0.1 mm to 5 mm in diameter, configured to optimize load distribution and bone ingrowth.
The bone void filler of claim 200, wherein the material is provided in pre-formed shapes including cylinders, blocks, and wedges, designed to optimize mechanical loading in specific anatomical applications.
The bone void filler further comprising a resorbable carrier selected from the group consisting of biocompatible liquids, hydrogels, and polymer solutions, suitable for injectable or putty preparations.
The bone void filler wherein the material is configured for use in conjunction with standard orthopedic fixation hardware in large segmental defects.
The bone void filler further comprising one or more therapeutic agents selected from the group consisting of antibiotics, analgesics, and growth factors, with controlled release properties.
The bone void filler wherein the material exhibits an interconnected porous structure with pore sizes ranging from 1 to 500 μm, optimized for vascular ingrowth and bone formation.

These and other objects and advantages of the invention will be apparent from the following description, the accompanying drawings and the appended claims.

### BRIEF DESCRIPTION OF THE ACCOMPANYING DRAWINGS

One or more exemplary embodiment(s) of the present disclosure is set forth in the following description, is shown in the drawings and is particularly and distinctly pointed out and set forth in the appended claims. The accompanying drawings, which are incorporated in and constitute a part of the specification, illustrate various example configurations and methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 is an exemplary depiction of cross-linked acrylic chains surrounding and stability a calcium phosphate particle of the calcium acrylic modified polymer.

FIG. 2 is an exemplary operational view of the calcium acrylic modified polymer being used in a long-bone defect;

FIG. 3 is an exemplary depictions of various shapes in which the calcium acrylic modified polymer may be shaped;

FIG. 4 is an exemplary depiction of a porous implant;

FIG. 5 is an exemplary depiction of a drug-eluting implant;

FIG. 6 is a flowchart depiction a method of treating a bone void;

FIG. 7 is a flowchart depiction a method of treating a large bone defect on a patient;

FIG. 8 is front view of a dental implant, where at least a portion of the dental implant is made of a calcium acrylic modified polymer;

FIG. 9 is a cross-sectional view of a portion of the dental implant body shown a microporosity structure extending from an outer surface to an internal structure of the dental implant body; and

FIG. 10 is an enlarged view of the microporosity structure.

Similar numbers refer to similar parts throughout the drawings.

### DETAILED DESCRIPTION OF THE PREFERRED EMBODIMENTS

This invention is based, at least in part, on the discovery of a bioactive composite including an osteo conductive ceramic compound dispersed within an acrylic polymer matrix including one or more polymerized acrylic compounds. Bones typically become damaged or otherwise sustain defects through trauma, surgery, and other causes and therefore require treatment including the use of bone void fillers to provide a scaffolding or support for bone regeneration. While a variety of synthetic, natural, and composite bone void fillers have been used in medical applications, the use of a bone void filler including a bioactive composite including an osteoconductive ceramic compound dispersed within an acrylic polymer matrix including one or more polymerized acrylic compounds has yielded unexpected benefits when used in medical applications.

### Material Composition and Properties

The base material consists of a calcium acrylic modified polymer (CAMP) matrix integrated with bioactive calcium phosphate components. Optional reinforcement materials may be incorporated for enhanced mechanical properties. The material can be manufactured in multiple physical forms, including injectable liquid/gel preparations, moldable putty, pre-formed shapes such as cylinders, blocks, and wedges, granules ranging from 0.1 mm to 5 mm, and custom-designed geometries for specific anatomical requirements.

The mechanical properties of the material include compressive strength ranging from 10-100 MPa and fatigue resistance capable of withstanding over 1 million cycles at physiological loading conditions. The elastic modulus is designed to match natural bone, with shape-specific load optimization achieved through internal architecture.

The biological properties include an osteoconductive surface promoting direct bone apposition, interconnected porosity ranging from 1-500 μm facilitating vascularization, surface micro-texture optimized for cell attachment, and controlled degradation rate matching bone ingrowth.

### Carrier Systems

Injectable formulations utilize biocompatible liquid carriers, resorbable polymer solutions, hydrogel-based systems, and temperature-sensitive carriers. Putty preparations incorporate cohesive carriers maintaining granule position, with moldable consistency and controlled setting properties.

### Drug Delivery Capabilities

The material can be loaded with antimicrobial agents for local antibiotic delivery, including but not limited to gentamicin, vancomycin, and tobramycin, with sustained release profiles and customizable drug loading based on clinical needs. Pain management capabilities include analgesic incorporation such as bupivacaine and morphine, with controlled release for post-operative pain management. Growth factors such as BMP-2, PDGF, and TGF-β, along with stem cell attractants and angiogenic factors, can be incorporated to enhance healing.

### Clinical Applications

In trauma applications, the material is suitable for long bone defects, metaphyseal voids, articular surface support, and integration with standard fixation hardware. Oncological applications include post-tumor resection reconstruction, large segmental defects, and prophylactic strengthening of metastatic lesions. For infection cases, the material can be used in two-stage revision procedures with local antibiotic delivery and dead space management. Spinal applications include interbody fusion procedures, vertebral body replacement, and posterior fusion applications.

### Bioactive Composites

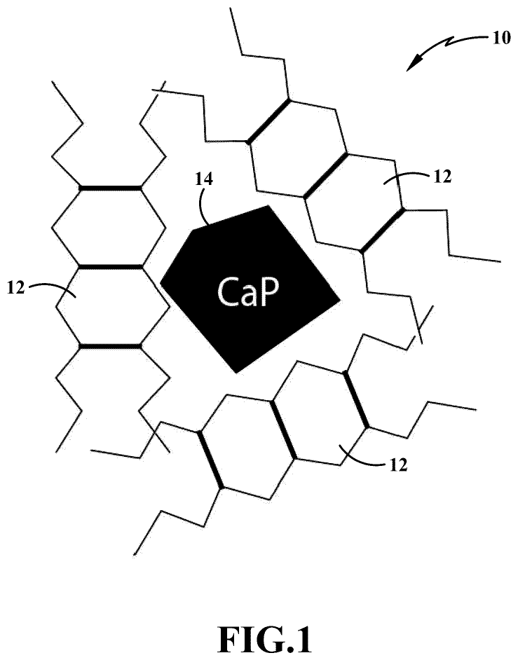

Bioactive composites of one or more embodiments of the present invention, which may also be referred to as ceramic acrylic modified polymer (“CAMP”) material, may be described with reference to FIG. 1.

Dental implants made from CAMP are designed to enhance osseointegration by promoting faster bone growth into the implant's porous structure. The surface microporosity of CAMP allows for greater interaction between the bone and implant, resulting in quicker healing times and stronger integration. The material also maintains long-term stability, minimizing the risk of implant failure due to loosening or infection.

CAMP provides a novel alternative to titanium, with the ability to mimic bone structure, promote healing, and integrate faster than current materials.

As shown in FIG. 1, bioactive composite 10 is shown in the form of a polymer matrix of a plurality of polymerized acrylic compounds 12 including an osteoconductive compound 14 dispersed within the plurality of polymerized acrylic compounds 12. As shown in FIG. 1, the plurality of polymerized acrylic compounds 12 are a plurality of crosslinked acrylic chains and osteoconductive compound 14 is a calcium phosphate compound.

The skilled person understands that the plurality of polymerized acrylic compounds includes polymers prepared from acrylate monomers and are notable for their resistance to breakage and elasticity. The polymerized acrylic compounds employed in the present invention include those that are biocompatible and demonstrate good adhesion to substrates including the surfaces of bones. Many polymerized acrylic compounds are known to those skilled in the art, and practice of this invention is not necessarily limited by the composition of the plurality of polymerized acrylic compounds. Known polymerized acrylic compounds that may be employed in practicing the present invention include, but are not limited to, compounds such as poly methyl methacrylate, ethylene glycol dimethacrylate (EGDMA), triethylene glycol dimethacrylate (TEGDMA), poly(ethylene glycol) dimethacrylate (PEGDMA), N,N′-methylenebisacrylamide (MBA), allyl methacrylate (AMA), urethane dimethacrylate (UDMA), bisphenol A glycerolate dimethacrylate (Bis-GMA), glyceryl dimethacrylate (GDMA), and mixtures thereof. The skilled person understands that the selection of these polymerized acrylic compounds may be tailored to adjust parameters. The amount of acrylic and polymer is larger than the amount of bioactive material because it strengthens the implants. In general, 2-20% of the compound is bioactive material and 80-98% is calcium phosphate or osteoconductive material.

The skilled person understands that osteoconductive ceramic compounds generally include those compounds used to facilitate bone regeneration and growth through the process of osteoconduction. The osteoconductive ceramic compounds employed in the present invention include those that are biocompatible and demonstrate support and scaffolding for new bone growth. Many osteoconductive ceramic compounds are known to those skilled in the art, and practice of this invention is not necessarily limited by the composition of the osteoconductive ceramic compounds. Known osteoconductive ceramic compounds that may be employed in practicing the present invention include, but are not limited to, compounds such as hydroxyapatite (HA, Ca₁₀(PO₄)₆(OH)₂), beta-tricalcium phosphate (β-TCP, Ca₃(PO₄)₂), biphasic calcium phosphate (BCP, combination of HA and β-TCP), calcium sulfate (CaSO₄), tetracalcium phosphate, octacalcium phosphate, bioactive glasses, silicate-based bioactive glasses, borate-based bioactive glasses, glass-ceramics: calcium phosphate and an acrylic polymer matrix. The preferred compound and components will depend in part on modules of elasticity, rigidity and duality. The skilled person understands that the selection of these osteoconductive ceramic compounds may be tailored to adjust parameters. As mentioned earlier, 2-20% of the compound is bioactive material and 80-98% is calcium phosphate or osteoconductive material.

Embodiments of the present invention advantageously may be bioactive in order to promote bone cell adhesion, proliferation, and differentiation by releasing calcium and/or phosphate ions into the tissue surrounding the bone defect being treated with the bioactive composite.

### Other Constituents of Bioactive Composites

The bioactive composite may further include other constituents and therapeutic agents. Those of ordinary skill the art appreciate that the bioactive composite may advantageously be biocompatible and resist inflammation or immune rejection when implanted into a patient's body. In order to do so, embodiments of the present invention advantageously have the capability to deliver drugs and other therapeutic agents. Examples of therapeutic agents suitable for use in bioactive composites according to the present invention include antibiotics, analgesics, growth factors, and each of these agents may further include controlled release properties. Many therapeutic agents are known in the art, and practice of this invention is not necessarily limited by the inclusion of any specific therapeutic agent.

In some applications, the bioactive composite may be loaded with one or more antimicrobial agent for local antibiotic delivery. Those of ordinary skill in the art understand that antimicrobial agents to prevent infection at a surgical sites, and practice of the present invention is not necessarily limited by the inclusion of any specific antimicrobial agent. Known antibiotic agents that may be employed in practicing the present invention include, but are not limited to, agents such as gentamicin, vancomycin and tobramycin. Antimicrobial agents used in embodiments of the present invention may have sustained release profiles and customizable drug loading based on clinical needs.

The bioactive composite can be used for pain management. Those of ordinary skill in the art understand that pain management can be achieved through the inclusion of analgesic agents in embodiments of bioactive composites and further practice of the present invention is not necessarily limited by the inclusion of any specific analgesic agent. Known analgesic agents that may be employed in practicing the present invention include, but are not limited to, agents such as bupivacaine and morphine. The analgesic may be controlled release for post-operative pain management.

As suggested above, the bioactive composites may be modified with bioactive agent selected from the group consisting of growth factors, stem cell attractant, and osteogenic proteins to enhance bone healing. Exemplary growth factors suitable for use in the present invention include, but are not limited to, BMP-2, PDGF, and TGF-β. Those of ordinary skill in the art understand that growth factors along with stem cell attractants and angiogenic factors can be incorporated to enhance healing without affecting practice of the present invention.

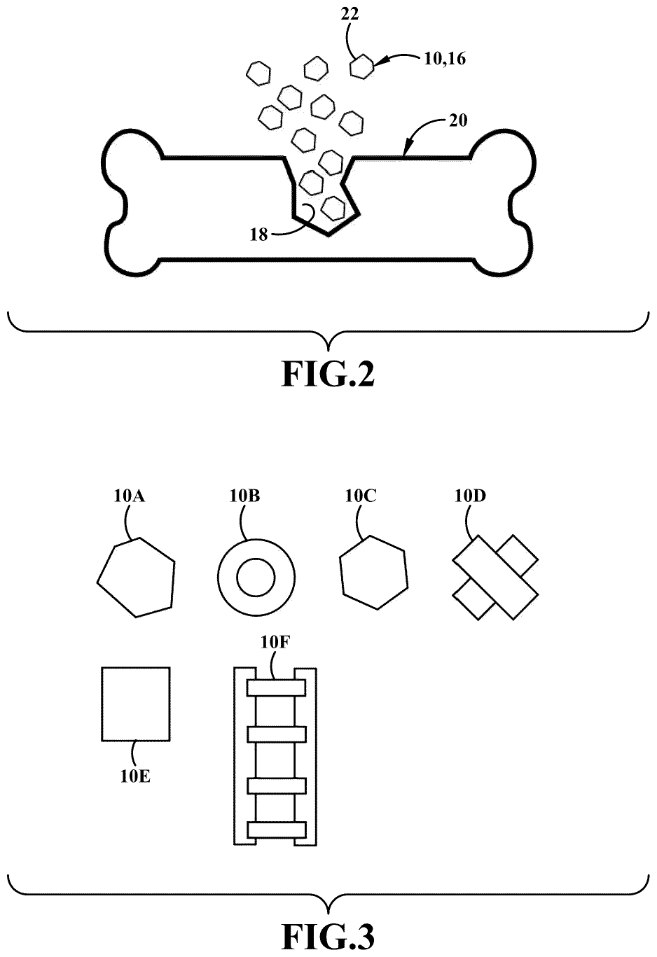

The bioactive composites may include at least one reinforcement material to allow for bioactive composite to have enhanced mechanical properties. As shown in FIG. 2 shows an exemplary use of bioactive composite 10 in the form of bone void filler 16 to fill the bone void or a long-bone defect 18 within bone 20. Bioactive composite 10 provides a scaffold, which will allow for bone 20 to regrow along the scaffold. Bioactive composite 10 is capable of maintaining the structural support necessary for bone 20 to regrow by maintaining mechanical integrity until bone 20 has regrown or regenerated within the bone void 18. The combination of the acrylic polymer matrix and osteoconductive ceramic compound allow for bioactive composites according to the present invention to maintain high strength while providing bioactive functionality. This feature advantageously provides for embodiments of the present invention to be employed at bone defects in load-bearing locations, such as long-bone defect 18 shown in FIG. 2.

### Characteristics of Bioactive Composites

The bioactive composites can be manufacturable in a variety of physical forms. Examples of these physical forms include granules, pre-formed shapes, custom-designed geometries, injectable liquid preparations, injectable gel preparations, and moldable putties. In embodiments manufactured as granules, the size of the granules is from about 0.1 mm to about 5 mm.

FIG. 3 depicts six different exemplary embodiments of bioactive composites according to the present invention. First shape 10A is a bioactive composite in the shape of an irregular hexagonal body. Second shape 10B is a bioactive composite in the shape of spherically or cylindrically shaped body. Third shape 10C is a bioactive composite in the shape of a regular hexagonal body. Fourth shape 10D is a bioactive composite in the shape of a three-dimensional X-shaped body. Fifth shape 10E is a bioactive composite in the shape of a planar body. Sixth shape 10F is a bioactive composite in the shape of a ladder-like body. Many suitable geometries for bioactive composites are known to those skilled in the art, and practice of this invention is not necessarily limited by the specific geometry of the bioactive composite. Bioactive composites according to the present invention may advantageously be custom-designed to have specific geometries useful for specific anatomical applications, including optimizing mechanical loading in the specific anatomical application.

The acrylic polymer matrix of the bioactive composite may be cross-linked to include one or more radiation-induced crosslinking, temperature-induced crosslinking, solution polymerization, di-ally linking, or chemical crosslinking.

Bioactive composites in any form advantageously maintain a high compressive strength. In one or more embodiments, the bioactive composite has a compressive strength of 10 MPa or greater, in other embodiments, a compressive strength of 25 MPa or greater, in other embodiments, a compressive strength of 50 MPa or greater, in other embodiments, a compressive strength of 75 MPa or greater, and yet in other embodiments, a compressive strength of 100 MPa or greater. In one or more embodiments, the bioactive composite has a compressive strength of from about 10 MPa to about 100 MPa.

Bioactive composites in any form advantageously are manufactured with a fatigue resistance capable of maintaining the stress or strain strength of over 1 million cycles at physiological loading conditions, such as compression, torsion, shar, static and fatigue. Further, Bioactive composites in any form advantageously are designed and manufactured to maintain an elastic modulus similar to natural bone. The skilled person understands that the internal architecture of embodiments of the bioactive composite may be configured to achieve shape-specific load optimization.

The bioactive composites may be radiopaque to allow for the bioactive composite to be monitored via radiographic imaging when used to treat a bone defect or void. The placement of the bioactive composite and bone regeneration at the site of a defect are advantageously monitored by the radiographic imaging.

The surfaces of bioactive composites may include various biological properties, including osteoconductive surfaces to promote direct bone apposition. As shown in FIG. 2, osteoconductive surface 22 is present to promote direct bone apposition to bone 20 at the site of bone defect 18.

Other biological properties of bioactive composites further include an interconnected porous structure of a plurality of pores. The interconnected plurality of pores is formed as the bioactive composite hardens or cures. In one or more embodiments, the interconnected plurality of pores of the hardened bioactive composite is characterized by an average pore diameter of each pore of the plurality of pores of from about 1 μm to about 500 μm, and in other embodiments of from about 1 μm to about 50 μm. The interconnected porosity advantageously facilitates vascularization of the bioactive composite at the defect and/or bone void and the surrounding bone. Said vascularization advantageously allows the new bone cells to infiltrate into the scaffold or support provided by the presence of the bioactive composite. Without wishing to be bound by theory it is believed that an average pore diameter of each pore of the plurality of pores of from about 1 μm and about 50 μm facilitates bone cell ingrowth and nutrient exchange.

The surface of the bioactive composite may be modified to include a micro-texture. Without wishing to be bound by theory it is believed that the surface including a micro-texture optimizes cellular adhesion during bone regrowth.

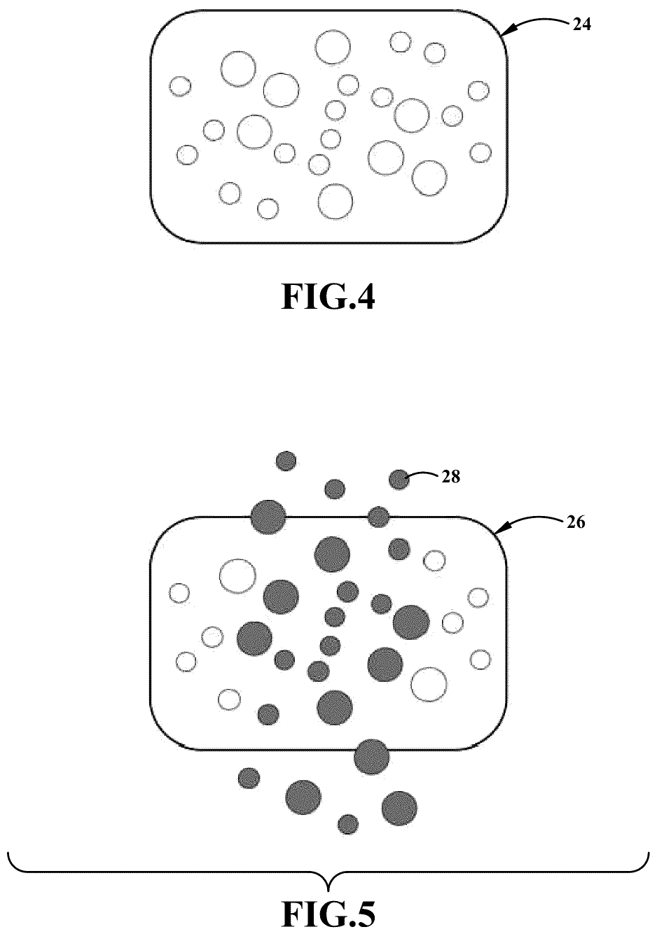

FIG. 4 shows an exemplary implant 24 which can be made of bioactive composites according to the present invention. Implant 24 includes a porous network including a pore structure and interconnectivity within the bioactive composite used to form implant 24.

FIG. 5 shows an exemplary implant 26 including an embodiment of the bioactive composite according to the present invention. Implant 26 is drug-eluting as shown by the plurality of drug particles 28 (e.g. therapeutic agents) diffusing from the implant 26.

Additionally, the degradation rate of the bioactive composite, and specifically the acrylic polymer matrix, may be tailored to a specific therapeutic application. As suggested above, bioactive composites according to the present invention provide a scaffolding through the acrylic polymer matrix to facilitate bone growth. Thus, a controlled degradation rate is designed to match a patient's bone in growth through the employment of bioresorbable fibers or particles to further enhance mechanical properties and ensure a controlled degradation profile. In one or more embodiments, the acrylic polymer matrix is cross-linked and has a stable structure (e.g. not degraded) period of 3 months or longer, in other embodiments 6 months or longer, in other embodiments 9 months or longer, and yet in other embodiments 12 months or longer.

### Applications of Bioactive Composites

As suggested above, bioactive composites according to embodiments of the present invention have a variety of uses, specifically in a variety of medical applications. Bioactive composites according to the present invention are suitable to be used in conjunction with a standard orthopedic fixation hardware that is known in the art for treating for example a large segmental defect or other similar medical applications. Specifically, bioactive composites according to the present invention are suitable for use in various medical applications such as orthopedic trauma, oncological tumor resections, or other applications which require bone reconstruction.

For example, bioactive composites according to the present invention may be used to treat a tibial plateau fracture. In the tibial plateau fracture, the bioactive composite is integrated with a standard plating system and progressive bone incorporation therapies over 12 months. The bioactive composite maintains its integrity over the 12 month period while supporting bone regeneration. The bone regeneration was confirmed using radiographical evidence to show the progressive incorporation and remodeling of the bone.

In a further example involving a case study of femoral diaphyseal reconstruction following a tumor resection, bioactive composites according to embodiments of the present invention were deployed in large segment replacement and integrated with intramedullary fixation. The progress of the bone regeneration, incorporation, and degradation over time was monitored by radiographic imaging and monitoring the bioactive composite through its radiopaque properties.

In yet another example involving a spinal fusion, embodiments of bioactive composites according to the present invention were employed in an interbody fusion procedure. The bioactive composite demonstrated sufficient compressive strength and fatigue resistance and was therefore suitable for vertebral body loading conditions.

In an additional example, embodiments of bioactive composites according to the present invention including antibiotic-loading material were used to successfully treat an infected non-union bone. The bioactive composite with the local antibiotic delivery provided sustained therapeutic levels while maintaining the mechanical properties of the bioactive composite.

In orthopedic trauma applications, the bioactive composites according to the present invention are suitable for a variety of cases, such as long bone defects, metaphyseal voids, articular surface support, and integration with standard fixation hardware. It will be understood that the bioactive composites according to the present invention may be used in any number of orthopedic trauma cases.

In oncological applications, the bioactive composites according to the present invention are suitable for post-tumor resection reconstruction, large segmental defect, and prophylactic strengthening of metastatic lesions. It will be understood that the bioactive composites according to the present invention may be used in any number of oncological cases which require bone reconstruction.

As suggested above, the bioactive composites according to the present invention may also be used for the treatment of infections. In these infection cases, the bioactive composite can be used in a two-stage revision procedure with local antibiotic delivery and dead space management. Bioactive composites according to the present invention may further be used for spinal applications, such as interbody fusion procedures, vertebral body replacement, and posterior fusion applications.

It will be understood that the bioactive composites according to the present invention may be used in any number of application which require a bone filler and aforementioned applications of bioactive composites according to the present invention are not exhaustive in nature.

In one or more embodiments, the bioactive composites according to the present invention are capable of forming a cohesive bond with the bone tissue surrounding the area in which bioactive composites are being placed. The cohesive bond that is formed prevents migration or displacement of the bioactive composite after implantation.

Additionally, the bioactive composites according to the present invention may be designed for use in pediatric patients. When bioactive composites according to the present invention are used in pediatric patients, the bioactive composites provide structural support while allowing for the pediatric patient's natural bone to grow and develop over time.

### Injectable Bone Void Filler Compositions

The person of ordinary skill in the art readily appreciates that bioactive composites according to the present invention may be deployed in the form of an injectable bone void filler composition, which may also be referred to as injectable formulations of bioactive composites. The injectable compositions of bioactive composites according to the present invention utilize biocompatible liquid carriers, resorbable polymer solutions, hydrogel-based systems, and temperature-sensitive carriers.

The injectable formulations of bioactive composites according to the present invention may be designed to be injectable or flowable within a carrier. Known carriers that may be employed in practicing the present invention include, but are not limited to, hyaluronic acid, collagen solutions, chitosan, sodium alginate, gelatin, methylcellulose, carboxymethylcellulose (CMC), polyethylene glycol (PEG) solutions, Pluronic F-127 (poloxamer 407), poly(vinyl alcohol) (PVA), poly(N-isopropylacrylamide) (PNIPAM), PEG-PCL copolymers, poly(lactide-co-glycolide) (PLGA) solutions, polycaprolactone (PCL) solutions, poly(L-lactide) (PLLA) solutions, poly(D,L-lactide) (PDLLA) solutions, poloxamer-based hydrogels, chitosan derivatives, glycerol, dextran, beta-cyclodextrin solutions, hydroxypropyl methylcellulose (HPMC), and phosphate buffered saline (PBS) with thickening agents. The skilled person appreciates that injectable formulations of bioactive composites according to the present invention may employ any number of carriers greater or smaller. As mentioned earlier, 2-20% of the compound is bioactive material and 80-98% is calcium phosphate or osteoconductive material.

The injectable formulations of bioactive composites according to the present invention may be formulated to exhibit thixotropic properties in some embodiments to advantageously facilitate injection and flow into the bone void or defect, especially irregularly shaped bone voids.

The injectable formulations of bioactive composites according to the present invention may be characterized by a set time, wherein after a period of time the bioactive composite provides mechanical stability to the bone void or defect. In one or more embodiments the set time of the injectable formulation of bioactive composite is 30 minutes or less, in other embodiments 25 minutes or less, in other embodiments 20 minutes or less, in other embodiments 15 minutes or less, in other embodiments 10 minutes or less, and in other embodiments 5 minutes or less. In one or more embodiments, the set time of the injectable formulation of bioactive composite from about 5 minutes to about 30 minutes.

As suggested above, bioactive composites according to the present invention may be prepared as putty or paste. In or more embodiments, putty preparations of bioactive composites according to the present invention incorporate cohesive carriers while maintaining a plurality of granules and provide moldable consistency and controlled setting properties. In these and other embodiments, the bioactive composite further includes a resorbable carrier which is suitable for injectable or putty preparations, and where the resorbable carrier is selected from the group consisting of biocompatible liquids, hydrogels, and polymer solutions.

### Methods of Structurally Supporting a Bone Defect

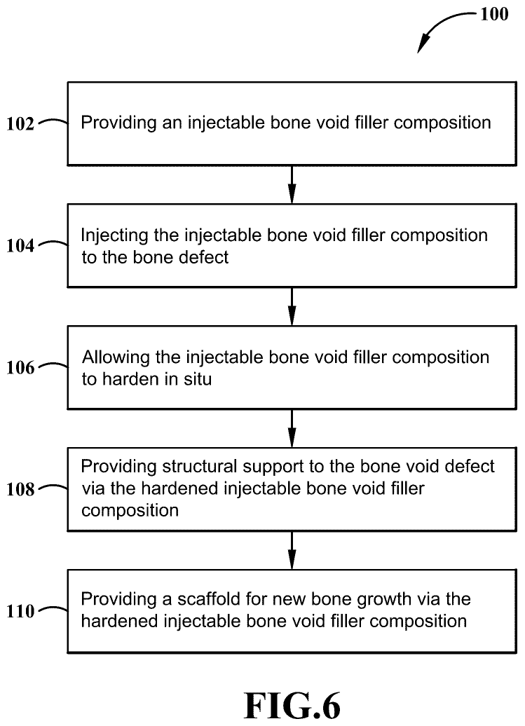

Referring now to FIG. 6, a method of structurally supporting a bone defect including defects formed from trauma and surgery is provided. Method 100 includes step 102 providing an injectable bone void filler composition 102, where the injectable bone void filler composition comprises a bioactive composite and a carrier, wherein the bioactive composite includes an osteoconductive ceramic compound dispersed within an acrylic polymer matrix, wherein the acrylic polymer matrix comprises one or more polymerized acrylic compounds. Method 100 further includes step 104 of injecting the injectable bone void filler composition to the bone defect. Method 100 next includes step 106 of allowing the injectable bone void filler composition to harden in situ. Method 100 includes step 108 of providing structural support to the bone void defect via the hardened injectable bone void filler composition. Finally, method 100 includes step 110 of providing a scaffold for new bone growth via the hardened injectable bone void filler composition. The scaffold is then used to allow for new bone growth.

Again and as mentioned earlier, 2-20% of the compound is bioactive material and 80-98% is calcium phosphate or osteoconductive material.

The step of injecting the injectable bone void filler composition to the bone defect may include a minimally invasive procedure to reduce a recovery time of the patient and minimize tissue damage to the patient.

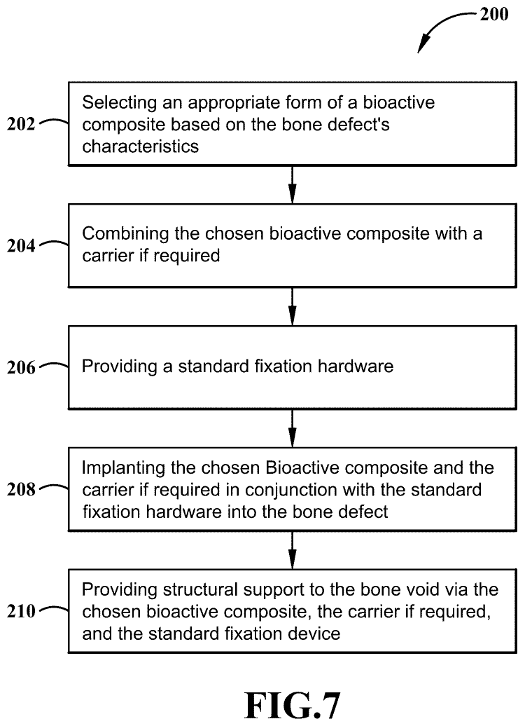

Referring now to FIG. 7, the method 200 includes step 202 of selecting an appropriate form of a bioactive composite based on the bone defect's characteristics. Method 200 further includes step 204 of combining the chosen bioactive composite with a carrier if required. Method further includes step 206 of providing a standard fixation hardware. Method 200 further includes step 208 of implanting the chosen Bioactive composite and the carrier if required in conjunction with the standard fixation hardware into the bone defect. Finally, method 200 includes the step of providing structural support to the bone void via the chosen bioactive composite, the carrier if required, and the standard fixation device. The chosen bioactive composite and the carrier, if required, provide long-term biological integration into the patient.

### Dental Implants

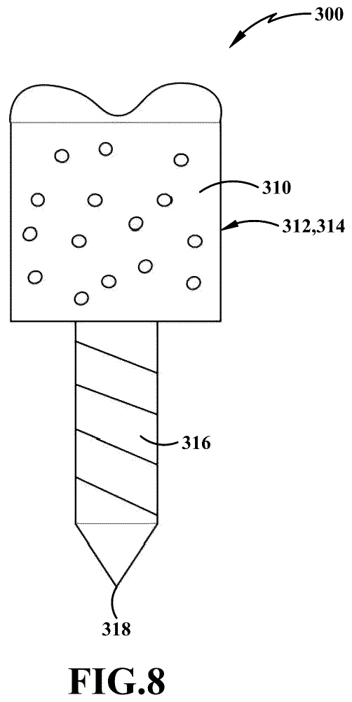

An example of an implant using systems and methods will now be described. In this illustration, a dental implant 300 according to an embodiment of the present invention as shown in FIG. 8. Dental implant 300 is made from a bioactive composite 310, which will be discussed in more detail below. Dental implant 300 is configured for insertion into a bone tissue of the patient. Bioactive composite 310 is similar to bioactive composite 10. Dental implant 300 is designed to enhance osseointegration by promoting faster bone growth into a porous structure of dental implant 300. A porous structure of the bioactive composite allows for greater interaction between a bone (not shown) and dental implant 300. The interaction between the bone and dental implant 300 allows for quicker healing times for a patient and stronger integration between the bone and dental implant 300.

It will be understood that although dental implant 300 is described and shown herein as a single tooth implant, dental implants according to other embodiments of the present invention may be any type of implant used in dental, orthopedic, or surgical applications.

In some embodiments, the bioactive composites used in dental implants maintain long-term stability similar to the bioactive composites described above. The long-term stability of the bioactive composite minimizes the risk of the dental implant failing due to loosening, infection, or other similar situations. The surface porosity of the bioactive composite allows for greater interaction between the bone and a dental implant, which results in quicker healing times and stronger integration for the patient. The bioactive composite is further desirable compared to the current materials for dental implants, such as titanium, due to the bioactive composite's ability to mimic bone structure, promote healing, and integrate with a patient's natural body quicker than current materials.

The bioactive composites used in dental implants can include one or more polymerized acrylic compounds in an acrylic polymer matrix similar to the one or more polymerized acrylic compounds in an acrylic polymer matrix of the bioactive composites described above. The bioactive composite further includes at least one osteoconductive ceramic compound similar to structural support to the bone void via the chosen the bioactive composite, the carrier if required, and the standard fixation device 14 of the bioactive composite. Specifically, the plurality of acrylic components of the bioactive composite are polymerized acrylic compounded to include at least one osteoconductive ceramic compound. the bioactive composite comprises one or more osteoconductive ceramics dispersed within the polymerized acrylic compounds. The one or more osteoconductive ceramics includes one or more of calcium phosphate, tricalcium phosphate, hydroxyapatite, or bioglass. The one or more osteoconductive ceramics are integrated into an acrylic polymer matrix similar to the bioactive composite 10 shown in FIG. 1. the bioactive composite provides bioactivity and mechanical properties that mimic natural bone.

The bioactive composites used in dental implants may be formulated to promote bone healing by enhancing cellular adhesion, proliferation, and differentiation of osteoblasts on the implant surface. the bioactive composite is further formulated to include osteoblasts-medicated surface remodeling and bone formation to a depth of greater than 1 mm form an implant surface.

Further, the bioactive composites used in dental implants may be biocompatible. The bioactive composite is designed to reduce the risk or peri-implantitis. The risk of peri-implantitis is reduced by providing long-term stability of the bioactive composite and the dental implant. The risk of peri-implantitis is further reduced by the bioactive composite's and the dental implant's resistance to a bacterial colonization.

Also, the bioactive composites used in dental implants may be modified to further reduce the risk of infections by including an analgesic or an antimicrobial agent. The analgesic or the antimicrobial agent may be silver ions or antibiotics.

Generally, the bioactive composites used in dental implants are designed to maintain long-term stability and long-term structural stability. In these and other embodiments, the bioactive composite is further designed to be resistant to degradation or wear under normal physiological loads experienced in an oral cavity.

Further, the bioactive composites used in dental implants have a long-term stability that is achieved by the bioactive composite being engineered to have a modulus of elasticity that is similar to the modulus elasticity of a natural bone. The similarity of the modulus of elasticity between the bioactive composite and the natural bone thereby minimizes a stress shielding effect and improving stability of the dental implant.

The bioactive composites used in dental implants are synthesized by incorporating osteoconductive ceramic into an acrylic polymer matrix. The synthesized bioactive composite has mechanical and biological properties which mimic the mechanical and biological properties of natural bone. Specifically, the bioactive composite has a radiodensity similar to natural bone. The similarity of the radiodensity allows for improved radiographic visibility of the bioactive composite during post-operative monitoring. The similarity of the radiodensity also helps ensure the dental implant is properly placed and has proper integration with the natural bone.

The bioactive composites used in dental implants may be designed to promote sustained ion release. The sustained ion release may include calcium ions or phosphate ions. The sustained ion release simulates mineralization around the dental implant.

The bioactive composites used in dental implants may be both compatible with other dental prosthetics and abutments. The bioactive composite and the dental implant may be both compatible with mechanical assemblies with metallic and polymer components. The compatibility of the bioactive composite and the dental implant enables integration of the bioactive composite or the dental implant with a variety of restorative dental procedures.

With further reference to FIG. 8, dental implant 300 includes body 312 formed of bioactive composite 310. Body 312 includes surface 314. Surface 314 of the body 312 is a porous surface. The porosity of surface 314 enhances osseointegration by promoting bone tissue ingrowth into the pores of surface 314 of dental implant 300.

In one or more embodiments, the porosity of the surface of the body of a dental implant is characterized by an average pore size of from about 1 μm to about 100 μm. The pore size of the surface can be optimized to promote bone tissue interaction and rapid osteointegration.

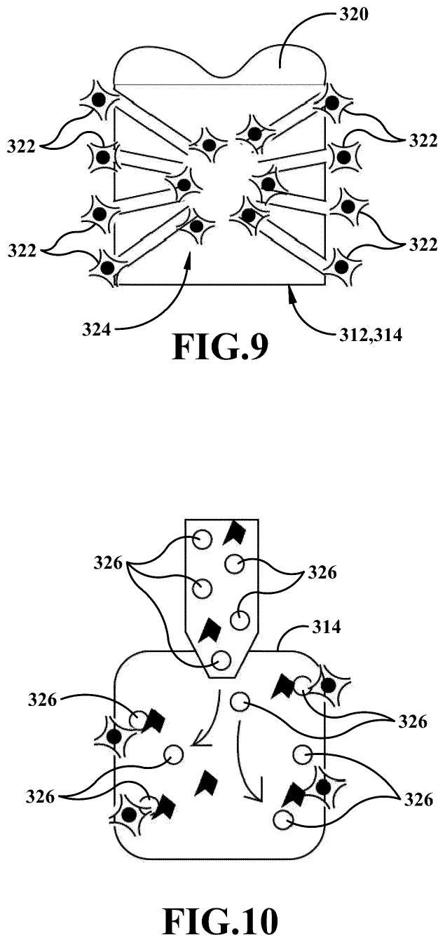

The body of the dental implant may be shaped and dimensioned to match an anatomical requirement of a dental implant site. As shown in FIG. 9 body 312 includes screw-like projection 316. Screw-like projection 316 includes threads to assist in the assertion of dental implant 300 into the bone, and specifically into a mandible of a patient. Screw-like projection 316 further includes apex 318 with a taper. Apex 318 and taper further assist in inserting dental implant 300 into the bone, and specifically into the mandible.

Referring now to FIG. 9, a cross-section of a portion of dental implant 300 is shown. The porosity of surface 314 is shown extending from surface 314 into internal structure 320 of dental implant 300. A plurality of interconnected pores 322 are shown extending from the porosity of surface 314 toward internal structure 320. The plurality of interconnected pores 322, collectively, define internal porous structure network 324. The internal porous structure network promotes osteoblast ingrowth.

Referring now to FIG. 10, the porosity of surface 314 includes pores 326. Pores 326 are created by gas entrapment during a surface modification technique to control the size and distribution of pores. The surface modification technique may be a molten acrylic phase, soluble porogen, or surface chemical etching.

### Methods of Fabricating Dental Implants

Another aspect of the present invention provides a method of manufacturing a dental implant. In these and other embodiments, the method of manufacturing a dental implant generally includes molding a body of the dental implant and treating a surface of the body to thereby form a plurality of surface pores in the surface.

The step of molding a body of the dental implant may include using a bioactive composite comprising an osteoconductive ceramic compound dispersed within an acrylic polymer matrix, wherein the acrylic polymer matrix comprises one or more polymerized acrylic compounds, and wherein the acrylic polymer matrix is crosslinked through one or more of radiation-induced crosslinking, temperature-induced crosslinking, solution polymerization, di-allyl linking, and chemical crosslinking.

The step of treating a surface of the body may include gas entrapment during a molten acrylic phase, soluble porogen, surface chemical etching, or other surface modification techniques to control the size and distribution of pores.

In one or more embodiments, methods of forming a dental implant further include applying a bioactive coating to the surface of the body, where the coating is selected from the group consisting of osteoconductive ceramics, collagen, and growth factors, to further enhance osseointegration and bone healing.

### ADDITIONAL CONSIDERATIONS

Various modifications and alterations that do not depart from the scope and spirit of this invention will become apparent to those skilled in the art. This invention is not to be duly limited to the illustrative embodiments set forth herein.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any flowchart and/or block diagrams in the Figures illustrate some exemplary architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. As another example, “at least one of: A, B, or B” is intended to cover A, B, C, A-B, A-C, B-C, and A-B-C, as well as any combination with multiple of the same item.

While components of the present disclosure are described herein in relation to each other, it is possible for one of the components disclosed herein to include inventive subject matter, if claimed alone or used alone. In keeping with the above example, if the disclosed embodiments teach the features of A and B, then there may be inventive subject matter in the combination of A and B, A alone, or B alone, unless otherwise stated herein.

As used herein in the specification and in the claims, the term “effecting” or a phrase or claim element beginning with the term “effecting” should be understood to mean to cause something to happen or to bring something about. For example, effecting an event to occur may be caused by actions of a first party even though a second party actually performed the event or had the event occur to the second party. Stated otherwise, effecting refers to one party giving another party the tools, objects, or resources to cause an event to occur. Thus, in this example a claim element of “effecting an event to occur” would mean that a first party is giving a second party the tools or resources needed for the second party to perform the event, however the affirmative single action is the responsibility of the first party to provide the tools or resources to cause said event to occur.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, “above”, “behind”, “in front of”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “lateral”, “transverse”, “longitudinal”, and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present disclosure.

An embodiment is an implementation or example of the present disclosure. Reference in the specification to “an embodiment,” “one embodiment,” “some embodiments,” “one particular embodiment,” “an exemplary embodiment,” or “other embodiments,” or the like, means that a particular feature, structure, or characteristic described in connection with the embodiments is included in at least some embodiments, but not necessarily all embodiments, of the invention. The various appearances “an embodiment,” “one embodiment,” “some embodiments,” “one particular embodiment,” “an exemplary embodiment,” or “other embodiments,” or the like, are not necessarily all referring to the same embodiments. Furthermore, the use of any and all examples or exemplary language (“e.g.,” “such as,” or the like) is intended merely to better illustrate or illuminate the embodiments and does not pose a limitation on the scope of that or those embodiments. No language in this specification should be construed as indicating any unclaimed element as essential to the practice of the disclosed embodiment.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element or “another” element, that does not preclude there being more than one of the additional element or the another element.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein. Further, recitation of ranges of values herein are not intended to be limiting, referring instead individually to any and all values falling within that range, unless otherwise indicated herein, and each separate value within such range is incorporated into the specification as if it were individually recited herein.

Additionally, the method of performing the present disclosure may occur in a sequence different than those described herein. Accordingly, no sequence of the method should be read as a limitation unless explicitly stated. It is recognizable that performing some of the steps of the method in a different order could achieve a similar result.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

To the extent that the present disclosure has utilized the term “invention” in various titles or sections of this specification, or in the context of those sections, this term has been included as required by the formatting requirements of word document submissions (i.e., docx submissions) pursuant the guidelines/requirements of the United States Patent and Trademark Office and shall not, in any manner, be considered a disavowal of any subject matter.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of various embodiments of the disclosure are examples and the disclosure is not limited to the exact details shown or described.

### ADDITIONAL EXAMPLES

Example 1: Trauma Application A clinical case demonstrates the material's use in a tibial plateau fracture, showing integration with standard plating systems and progressive bone incorporation over 12 months. The material maintains structural integrity while supporting bone regeneration, with radiographic evidence of progressive incorporation and remodeling.

Example 2: Oncologic Reconstruction A case study of femoral diaphyseal reconstruction following tumor resection demonstrates the material's capability in large segment replacement and integration with intramedullary fixation. The material's radiopaque properties allow for monitoring of incorporation and remodeling over time.

Example 3: Spinal Fusion Documentation of interbody fusion procedures demonstrates the material's load-bearing capabilities and fusion progression. The material's compressive strength and fatigue resistance prove suitable for vertebral body loading conditions.

Example 4: Infection Management A two-stage revision protocol incorporating antibiotic-loaded material demonstrates successful treatment of infected non-union. Local antibiotic delivery provides sustained therapeutic levels while maintaining mechanical properties.

### Advantages

The invention provides superior mechanical properties compared to existing bone void fillers, versatile delivery options, enhanced biological activity, drug delivery capabilities, compatibility with standard fixation systems, optimized handling characteristics, and controlled resorption profile.

### INDUSTRIAL APPLICABILITY

This invention provides significant advancement in the field of orthopedic surgery and bone reconstruction, offering a versatile solution for various clinical challenges while maintaining both mechanical and biological requirements for optimal bone healing. The material's versatility in form and function makes it suitable for a wide range of clinical applications, from small bone voids to large segmental defects.

Advantageously, another embodiment of this invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, apparatus and method herein described constitute preferred embodiments of this invention, it is to be understood that the invention is not limited to this precise system, apparatus and method, and that changes may be made therein without departing from the scope of the invention which is defined in the appended claims.

What is claimed is:

1. A bioactive composite comprising:
an osteoconductive ceramic compound dispersed within an acrylic polymer matrix;
wherein the acrylic polymer matrix comprises one or more polymerized acrylic compounds.

2. The bioactive composite of claim 1, wherein the one or more polymerized acrylic compounds are selected from the group consisting of poly methyl methacrylate, ethylene glycol dimethacrylate (EGDMA), triethylene glycol dimethacrylate (TEGDMA), poly(ethylene glycol) dimethacrylate (PEGDMA), N,N′-methylenebisacrylamide (MBA), allyl methacrylate (AMA), urethane dimethacrylate (UDMA), bisphenol A glycerolate dimethacrylate (Bis-GMA), glyceryl dimethacrylate (GDMA), and mixtures thereof.

3. The bioactive composite of claim 1, wherein the osteoconductive ceramic compound is selected from the group consisting of hydroxyapatite (HA, Ca₁₀(PO₄)₆(OH)₂), beta-tricalcium phosphate (β-TCP, Ca₃(PO₄)₂), biphasic calcium phosphate (BCP, combination of HA and β-TCP), calcium sulfate (CaSO₄), tetracalcium phosphate, octacalcium phosphate, bioactive glasses, silicate-based bioactive glasses, borate-based bioactive glasses, and combinations thereof.

4. The bioactive composite of claim 1, wherein the acrylic polymer matrix is cross-linked.

5. The bioactive composite of claim 4, wherein the cross-linked acrylic polymer matrix comprises a plurality of pores wherein an average diameter of each pore of the plurality of pores is from 1 μm to 500 μm.

6. The bioactive composite of claim 5, wherein the average diameter of each pore of the plurality of pores is from 1 μm to 50 μm.

7. The bioactive composite of claim 4, wherein the cross-linked acrylic polymer matrix structure is stable for a period of at least 3 months.

8. The bioactive composite of claim 1, wherein the bioactive composite has a compressive strength of 10 MPa or greater.

9. The bioactive composite of claim 1, wherein the bioactive composite is radiopaque.

10. The bioactive composite of claim 1, further comprising one or more bioactive agents selected from the group consisting of growth factors, stem cell attractants, osteogenic proteins, and combinations thereof.

11. The bioactive composite of claim 1, further comprising one or more of antimicrobial agents, analgesics, and growth factors, with and without controlled release properties.

12. An injectable bone void filler composition comprising:
the bioactive composite of claim 1 in a flowable form; and
a carrier.

13. The injectable bone void filler composition of claim 12, wherein the carrier is selected from the group consisting of one of hyaluronic acid, collagen solutions, chitosan, sodium alginate, gelatin, methylcellulose, carboxymethylcellulose (CMC), polyethylene glycol (PEG) solutions, Pluronic F-127 (poloxamer 407), poly(vinyl alcohol) (PVA), poly(N-isopropylacrylamide) (PNIPAM), PEG-PCL copolymers, poly(lactide-co-glycolide) (PLGA) solutions, polycaprolactone (PCL) solutions, poly(L-lactide) (PLLA) solutions, poly(D,L-lactide) (PDLLA) solutions, poloxamer-based hydrogels, chitosan derivatives, glycerol, dextran, beta-cyclodextrin solutions, hydroxypropyl methylcellulose (HPMC), phosphate buffered saline (PBS) with thickening agents, and combinations thereof.

14. The injectable bone void filler composition of claim 12, wherein the bioactive composite further comprises one or more bioresorbable components.

15. The injectable bone void filler composition of claim 12, wherein the injectable bone void filler is thixotropic.

16. The injectable bone void filler composition of claim 12, wherein the injectable bone void filler composition has a set time of from about 5 minutes to about 30 minutes.

17. A dental implant comprising:
a body comprising the bioactive composite of claim 1.

18. The dental implant of claim 17, wherein the body comprises a surface, wherein the surface includes a plurality of pores.

19. The dental implant of claim 18, wherein the body further comprises a bioactive coating over the surface, and wherein the bioactive coating is selected from the group consisting of osteoconductive ceramics, collagen, growth factors, and combinations thereof.

20. A method of structurally supporting a bone defect including defects formed from trauma and surgery, the method comprising:
providing an injectable bone void filler composition comprising:
an osteoconductive ceramic compound dispersed within an acrylic polymer matrix, wherein the acrylic polymer matrix comprises one or more polymerized acrylic compounds; and
a carrier;
injecting the injectable bone void filler composition to the bone defect and allowing the injectable bone void filler composition to harden in situ to thereby form a structural support to the bone.

21. A method of manufacturing a dental implant, the method comprising:
molding a body of the dental implant using a bioactive composite comprising an osteoconductive ceramic compound dispersed within an acrylic polymer matrix, wherein the acrylic polymer matrix comprises one or more polymerized acrylic compounds, and wherein the acrylic polymer matrix is crosslinked through one or more of radiation-induced crosslinking, temperature-induced crosslinking, solution polymerization, di-allyl linking, and chemical crosslinking;
treating a surface of the body to thereby form a plurality of surface pores in the surface.

22. A dental implant comprising:
a body formed from a calcium acrylic modified polymer (CAMP), comprising a polymerized acrylic compounded with calcium-containing ceramics wherein the implant is configured for insertion into bone tissue;
the surface of the body comprising microporosity to enhance osseointegration by promoting bone tissue ingrowth into the porous structure of the implant;
said acrylic comprising one or more of radiation-induced crosslinking, temperature-induced crosslinking, solution polymerization, di-allyl linking, or chemical crosslinking.

23. The dental implant of claim 22, wherein the surface microporosity has an average pore size between 1 μm and 100 μm to optimize bone tissue interaction and promote rapid osseointegration.

24. The dental implant of claim 22, wherein the CAMP material comprises one or more osteoconductive ceramics, including on or more of calcium phosphate, tricalcium phosphate, hydroxyapatite, or bioglass, integrated into an acrylic polymer matrix to provide bioactivity and mechanical properties that mimic natural bone.

25. The dental implant of claim 22, wherein the CAMP material is formulated to promote bone healing by enhancing cellular adhesion, proliferation, and differentiation of osteoblasts on the implant surface, including osteoblast-mediated surface remodeling and bone formation to a depth of greater than 1 mm from the implant surface.

26. The dental implant of claim 22, wherein the CAMP material is biocompatible and designed to reduce the risk of peri-implantitis by providing long-term stability and resistance to bacterial colonization.

27. The dental implant of claim 22, wherein the body of the implant is shaped and dimensioned to match the anatomical requirements of a dental implant site, including a screw-type design for insertion into the mandible.

28. The dental implant of claim 22, wherein the surface microporosity is created by gas entrapment during a molten acrylic phase, soluble porogen, surface chemical etching, or other surface modification techniques to control the size and distribution of pores.

29. The dental implant of claim 22, further comprising a bioactive coating or external surface of a multi-part assembly applied to the microporous surface, wherein the coating is selected from the group consisting of osteoconductive ceramics, collagen, and growth factors, to further enhance osseointegration and bone healing.

30. The dental implant of claim 22, wherein the CAMP material is modified to include analgesics or antimicrobial agents, such as silver ions or antibiotics.

31. The dental implant of claim 22, wherein the porosity of the surface extends into the internal structure of the implant, forming interconnected pores that allow for vascularization and enhanced bone ingrowth.

32. The dental implant of claim 22, wherein the CAMP material is engineered to provide a modulus of elasticity that closely matches that of natural bone, thereby minimizing stress shielding effects and improving implant stability.

33. The dental implant of claim 22, wherein the CAMP material is designed to maintain long-term structural stability and resist degradation or wear under normal physiological loads experienced in the oral cavity.

34. A method of manufacturing a dental implant, comprising:
molding a body of the implant from ceramic-acrylic modified polymer (CAMP);
introducing surface microporosity of the body of the implant to promote osseointegration,
wherein the CAMP material is designed to enhance bone tissue interaction and ensure faster healing and stronger integration compared to traditional implant materials.

35. The method of claim 34, wherein the surface microporosity is created by molten-state gas entrapment, porogen, or chemical etching techniques, thereby producing a porous structure optimized for bone ingrowth.

36. The method of claim 34, wherein the CAMP material is synthesized by incorporating osteoconductive ceramic into an acrylic polymer matrix, resulting in a composite material that mimics the mechanical and biological properties of bone.

37. The dental implant of claim 22, wherein the CAMP material has a radiodensity similar to bone, allowing for improved radiographic visibility during post-operative monitoring and ensuring proper implant placement and integration.

38. The dental implant of claim 22, wherein the CAMP material is designed to promote sustained ion release, including calcium and phosphate ions, to stimulate bone mineralization around the implant.

39. The dental implant of claim 22, wherein the CAMP material and/or component is compatible with other dental prosthetics and abutments, including mechanical assembly with metallic or polymer components, enabling integration into a variety of restorative dental procedures.

40. An injectable bone void filler comprising:
a ceramic acrylic modified polymer (CAMP) material;
wherein the CAMP material is configured to provide structural support to the bone void and serves as a scaffold for bone regrowth;
wherein the material maintains mechanical integrity until natural bone regenerates within the void;
said acrylic material comprising poly methyl methacrylate, Ethylene glycol dimethacrylate (EGDMA), Triethylene glycol dimethacrylate (TEGDMA), Poly(ethylene glycol) dimethacrylate (PEGDMA), N,N′-methylenebisacrylamide (MBA), Allyl methacrylate (AMA), Urethane dimethacrylate (UDMA), Bisphenol A glycerolate dimethacrylate (Bis-GMA), Glyceryl dimethacrylate (GDMA).

41. The bone void filler of claim 40, wherein the CAMP material comprises a combination of at least one of Hydroxyapatite (HA, Ca₁₀(PO₄)₆(OH)₂), Beta-tricalcium phosphate (β-TCP, Ca₃(PO₄)₂), Biphasic calcium phosphate (BCP, combination of HA and β-TCP), Calcium sulfate (CaSO₄), Tetracalcium phosphate, Octacalcium phosphate, Bioactive glasses, Silicate-based bioactive glasses, Borate-based bioactive glasses, Glass-ceramics: calcium phosphate and an acrylic polymer matrix, providing both bioactivity and strength suitable for load-bearing applications.

42. The bone void filler of claim 40, wherein the material is designed to be injectable or flowable within a carrier such as Hyaluronic acid, Collagen solutions, Chitosan, Sodium alginate, Gelatin, Methylcellulose, Carboxymethylcellulose (CMC), Polyethylene glycol (PEG) solutions, Pluronic F-127 (poloxamer 407), Poly(vinyl alcohol) (PVA), Poly(N-isopropylacrylamide) (PNIPAM), PEG-PCL copolymers, Poly(lactide-co-glycolide) (PLGA) solutions, Polycaprolactone (PCL) solutions, Poly(L-lactide) (PLLA) solutions, Poly(D,L-lactide) (PDLLA) solutions, Poloxamer-based hydrogels, chitosan derivatives, Glycerol, Dextran, Beta-cyclodextrin solutions, Hydroxypropyl methylcellulose (HPMC), and/or Phosphate buffered saline (PBS) with thickening agents.

43. The bone void filler of claim 40, wherein the material is bioactive and promotes bone cell adhesion, proliferation, and differentiation by releasing calcium and/or phosphate ions into the surrounding tissue.

44. The bone void filler of claim 40, wherein the material forms a porous structure upon hardening, allowing for vascularization and infiltration of new bone cells into the scaffold.

45. The bone void filler of claim 40, wherein the material is designed to gradually degrade over time as new bone tissue forms, ensuring the complete replacement of the filler by natural bone.

46. The bone void filler of claim 40, wherein the CAMP material is radiopaque, allowing for easy monitoring of the filler placement and bone regeneration using radiographic imaging.

47. The bone void filler of claim 40, wherein the CAMP material exhibits a compressive strength of at least 10 MPa, sufficient to provide mechanical support in load-bearing bone voids.

48. The bone void filler of claim 40, wherein the material is designed to maintain its structural integrity for a period of 3 to 12 months, providing temporary support until the bone fully regenerates.

49. The bone void filler of claim 40, wherein the material is modified with bioactive agents selected from the group consisting of growth factors, stem cell attractants, and osteogenic proteins to enhance bone healing.

50. The bone void filler of claim 40, wherein the CAMP material includes micropores with an average size between 1 μm and 50 μm to facilitate bone cell ingrowth and nutrient exchange.

51. The bone void filler of claim 40, wherein the material is suitable for use in orthopedic trauma, tumor resections, or other conditions requiring bone reconstruction.

52. A method for treating bone voids, comprising injecting a flowable CAMP-based material into a bone void and allowing the material to harden in situ, wherein the material provides structural support and serves as a scaffold for new bone growth.

53. The method of claim 52, wherein the CAMP material is injected through a minimally invasive surgical procedure to reduce patient recovery time and minimize tissue damage.

54. The bone void filler of claim 40, wherein the material is formulated to exhibit thixotropic properties, allowing for easy injection and flow into irregularly shaped bone voids.

55. The bone void filler of claim 40, wherein the material includes antimicrobial agents to prevent infection at the surgical site or analgesics.

56. The bone void filler of claim 40, wherein the CAMP material is combined with bioresorbable fibers or particles to further enhance mechanical properties and ensure a controlled degradation profile.

57. The bone void filler of claim 40, wherein the material is biocompatible and resists inflammation or immune rejection when implanted in a human body.

58. The bone void filler of claim 40, wherein the CAMP material is designed to set within a time frame of 5 to 30 minutes after injection, providing rapid mechanical stabilization to the bone void.

59. The bone void filler of claim 40, wherein the material is capable of forming a cohesive bond with the surrounding bone tissue, preventing migration or displacement of the filler after implantation.

60. The bone void filler of claim 40, wherein the material is designed for use in pediatric patients, providing structural support while allowing for natural bone growth and development over time.

61. A bone reconstruction system comprising a CAMP-based injectable bone void filler as described in claim 40 and an applicator configured to inject the flowable material into a bone void, wherein the system provides structural support and bioactivity for the treatment of bone defects caused by trauma or surgery.

62. The bone void filler of claim 40, wherein the material demonstrates fatigue resistance capable of withstanding cyclic loading of at least 1 million cycles under physiological loading conditions.

63. The bone void filler of claim 40, wherein the material is provided in the form of granules ranging from 0.1 mm to 5 mm in diameter, configured to optimize load distribution and bone ingrowth.

64. The bone void filler of claim 40, wherein the material is provided in pre-formed shapes including cylinders, blocks, and wedges, designed to optimize mechanical loading in specific anatomical applications.

65. The bone void filler of claim 40, further comprising a resorbable carrier selected from the group consisting of biocompatible liquids, hydrogels, and polymer solutions, suitable for injectable or putty preparations.

66. The bone void filler of claim 40, wherein the material is configured for use in conjunction with standard orthopedic fixation hardware in large segmental defects.

67. The bone void filler of claim 40, further comprising one or more therapeutic agents selected from the group consisting of antibiotics, analgesics, and growth factors, with controlled release properties.

68. The bone void filler of claim 40, wherein the material exhibits an interconnected porous structure with pore sizes ranging from 1 to 500 μm, optimized for vascular ingrowth and bone formation.

69. A method of treating large bone defects, comprising selecting an appropriate form of the CAMP material based on defect characteristics, combining the material with a carrier if required, and implanting the material in conjunction with standard fixation hardware, wherein the material provides both immediate mechanical stability and long-term biological integration.
